(19) United States
(12) Patent Application Publication
(10) Pub. No.: US 2002/0182595 A1
(43) Pub. Date: Dec. 5, 2002

(54) Method of identifying cellular regulators of adeno-associated virus (AAV)

(76) Inventors: Matthew D. Weitzman, La Jolla, CA (US); Anton J. Cathomen, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/135,984

(22) Filed: Apr. 29, 2002

Related U.S. Application Data
(60) Provisional application No. 60/286,951, filed on Apr. 27, 2001.

(51) Int. Cl. C12Q 1/70; C12Q 1/68

(52) U.S. Cl. 435/5; 435/6

(57) ABSTRACT

The present invention relates to a genetic screening assay to identify molecules that interact with a viral regulatory element. The viral regulatory element may be derived from an adeno associated virus (AAV), and may optionally contain at least one inverted terminal repeats (ITR) or one or more regions thereof. The construct containing the viral regulatory sequence is linked to a reporter gene so that the reporter gene will be expressed in the presence of proteins or other molecules that bind to the viral regulatory element. The assay is beneficial for analyzing molecules that bind to viral regulatory regions, and may be also useful as an assay kit to examine such interactions.

### RELATED APPLICATIONS

This application claims the benefit of priority of U.S. Provisional Application No. 60/286,951, filed Apr. 27, 2001, incorporated herein in its entirety.

### BACKGROUND OF THE INVENTION

Adeno-associated virus (“AAV”) is a helper-dependent DNA parvovirus which belongs to the genus Dependovirus. AAV requires co-infection with an unrelated helper virus, either adenovirus, a herpesvirus or vaccinia, in order for a productive infection to occur. In the absence of such co-infection, AAV establishes a latent state by insertion of its genome into a host cell chromosome. Subsequent infection by a helper virus rescues the integrated copy which can then replicate to produce infectious viral progeny. AAV has a wide host range and is able to replicate in cells from any species so long as there is also a successful co-infection of such cells with a suitable helper virus. AAV has not been associated with any human or animal disease and does not appear to alter the biological properties of the host cell upon integration. For a review of AAV, see, e.g., Muzyczka N. & Berns, K. I. (2001) Parvoviridae: The Viruses and Their Replication, in Fields Virology Vol. 2 (eds. Knipe, D. M. & Howley, P. M.), Lippincott Williams & Wilkins, Philadelphia, pp. 2327-2359. AAV has been used to infect cells in vivo and in vitro and thus, an understanding of AAV life cycle is important to and therapeutic methods. The AAV genome is composed of a linear, single-stranded DNA molecule which contains about 4675 to 4681 bases (Bems and Bohenzky, (1987) Advances in Virus Research (Academic Press, Inc.) 32:243-307; Srivastava A. et al. (1983) J Virol. 45(2):555-64.

The genome includes inverted terminal repeats (ITRs) at each end which function in cis as origins of DNA replication and as packaging signals for the virus. The ITRs are approximately 145 nucleotides in length. The internal non-repeated portion of the genome includes two large open reading frames, known as the AAV rep and cap regions, respectively. These regions code for the viral proteins involved in replication and packaging of the virion. In particular, a family of at least four viral proteins is synthesized from the AAV rep region and includes Rep 78, Rep 68, Rep 52 and Rep 40, named according to their apparent molecular weight. The AAV cap region encodes at least three proteins, VP1 , VP2 and VP3. For a detailed description of the AAV genome, see, e.g., Muzyczka, N. and Bems (2001) supra.

The large, non-structural proteins of AAV, Rep78 and Rep68, are required for replication (Ni, T. H. et al. (1994) J Virol 68, 1128-38; Ward, P. et al. (1994) J Virol 68, 6029-37; and Tratschin, J. D. et al. (1984) J Virol 51, 611-9), targeted integration (Weitzman, M. D. et al. (1994) Proc Natl Acad Sci U S A 91, 5808-12; Linden, R. M. et al.(1996) Proc Natl Acad Sci U S A 93, 7966-72; and Surosky, R. T. et al.(1997) J Virol 71, 7951-9) and rescue from the latent state (Mendelson, E. et al.(1988) Virology 166,154-65) by binding to specific cis-acting sequences, named Rep Recognition Sequence (RRS) or Rep Binding Element (RBE). The RRS consists of an imperfect GCTC repeating motif, which is found within the pre-integration locus AAVS 1, the viral inverted terminal repeats (ITRs) and the p5 promoter (Weitzman et al. (1994) supra; Linden et al. (1996) supra; and McCarty, D. M. et al.(1994) J Virol 68, 4988-97). Moreover, Rep78/68 proteins were shown to regulate their own expression by binding to the p5RRS (Kyosito, S. R. (1995) J Virol 69, 6787-96; Cathomen T et al.(2000) J Virol. 74(5):2372-82; Wonderling R. S. et al. (1997) Virology. 236(1):167-76; and Wang, X. S. & Srivastava, A. (1998) J Virol 72, 4811-8)). They are also involved in trans-regulation of the AAV p19 and p40 promoters (Pereira, D. J. & Muzyczka, N. (1997) J Virol 71, 1747-56; Yang, Q. et al. (1994) J Virol 68, 4847-56) as well as of some heterologous promoters to inhibit cell growth, cellular transformation and replication of other viruses see, e.g., Muzyczka, N. and Bems(2001) supra.

Activation of the AAV p5 promoter, which drives expression of the large Rep78/68 proteins, is a crucial step in the AAV life-cycle. In the absence of helper functions, the p5 promoter has been shown to be repressed in trans by the cellular transcription factor YY1 (Kyosito, S. R. (1995) J Virol 69, 6787-96; Wang, X. S. & Srivastava, A. (1998) J Virol 72, 4811-8; and Shi, Y. et al.(1991) Cell 67, 377-88) and autoregulated by the large Rep proteins (Beaton, A. et al. (1989) J Virol 63, 4450-4; Labow, M. A. et al. (1986) J Virol 60, 251-8). Repression in tissue culture is eliminated upon infection with adenovirus due to expression of E1A, which relieves YY1-mediated repression (Shi, Y. et al.(1991), supra). Transactivation of the AAV p19 and p40 promoters is another crucial step to productive infection (Pereira, D. J. et al. (1997) J Virol 71, 1079-88). It has been shown that Rep binding to the ITR-RRS transactivates all three AAV promoters in an enhancer-like fashion (id.).

The construction of recombinant AAV virions has been described. See, e.g., U.S. Pat. Nos. 5,173,414 and 5,139,941; International Publication Numbers WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 15 8:97-129; Kotin, R. M. (1994) Human Gene Therapy 5:793-801 and Grimm & Kleinschmidt, Human Gene Therapy (1999) 10:2445-50.

Recombinant AAV (rAAV) virions are generally produced in a suitable host cell that has been transfected with two constructs including an AAV vector plasmid and a Rep/Cap plasmid, whereby the host cell is thus capable of expressing the AAV proteins necessary for AAV replication and packaging. The host cell is then co-infected with an appropriate helper virus or Ad helper plasmids (Xiao et al. (1998) J. Virol. 72:2224-32) to provide necessary viral helper functions. AAV functions can be provided by transfecting the host cell with an AAV plasmid that includes the AAV rep and/or cap coding regions but which lacks the AAV ITRs. Accordingly, the plasmid can neither replicate nor package itself. A number of vectors that contain the rep coding region are known, including those vectors described in U.S. Pat. No. 5,139,941, having ATCC accession numbers 53222, 53223, 53224, 53225 and 53226. A number of vectors containing the cap coding region have also been described, including those vectors described in U.S. Pat. No. 5,139,941.

### SUMMARY OF THE INVENTION

In one embodiment, the invention provides a method for identifying molecules that interact with a viral regulatory element, involving the steps of contacting a nucleic acid having one or more viral regulatory elements with one or more molecules; and then detecting whether the molecules are bound to the viral regulatory elements. The viral regulatory element may include the sequence of an inverted terminal repeat (ITR) as depicted in the linear sequence shown in FIG. 1 (SEQ ID NO:8). The viral regulatory element may include (one or more copies, one, two, three, four, five or more), of a region of the viral ITR including regions A, A′, B B′, C, C′ and D (see FIG. 1). Regions are typically utilized as double-stranded base paired stem structures: stem A (A:A′), stem B (B:B′); stem C (C:C′). Region D remains single stranded. In one aspect of the invention a sub-region, the Rep Recognition Sequence (RRS) GAGCGAGCGAGCGCGC, (SEQ ID NO: 1) may be present in one, two, three, four, five or more copies and may occur in tandem Further, the viral regulatory element may be derived from other regions of the genomes of adeno-associated virus and other AAV serotypes in addition to AAV-2, including, but not limited to, AAV-1, AAV-3, AAV-4, AAV-5 and AAV-6.

In aspects of the invention the molecules may be proteins and in particular cellular proteins. In other aspects of the invention the molecules may be peptides, nucleic acids or small molecules.

In further aspects of the invention, the nucleic acid comprising the viral regulatory element may further contain a reporter gene positioned 3′ to the regulatory element, so that detection of the bound molecule is by detecting the product of the reporter gene. In one embodiment, the reporter gene encodes beta-galactosidase, and detection is by color change.

In other embodiments of the invention, the viral regulatory element may be operably linked with a nutritional reporter gene so that the bound molecule may be detected by the ability of a strain to grow under selective conditions. In some embodiments, the nutritional reporter gene is HIS3.

In some embodiments of the invention, the one or more viral regulatory elements may be integrated into a yeast genome.

In further embodiments, detecting whether the molecules are bound to the viral regulatory elements is done by a method selected from the group consisting of virus product formation, 2D gel electrophoresis, electrophoretic mobility shift assay, immunoprecipitation, bimolecular interaction assay (BIAcore), affinity chromatography and two-hybrid assay systems.

Cellular proteins may be produced from a cDNA library, which is introduced into a cell and expressed. The cellular proteins may be present in a cellular lysate. In another embodiment, the cellular proteins may be human. In addition, the cellular proteins may be obtained by isolating or purifying them from other cells.

In a further embodiment, the invention may be an assay kit that is used to analyze the interaction between molecules and a viral regulatory element. Such a kit would have a viral regulatory element and a reporter gene positioned 3′ to the regulatory element, so that the product of the reporter gene can be detected when a molecule is bound to the viral regulatory element. This type of kit may include an inverted terminal repeat (ITR), one to several copies of one or more regions or sub-regions of the ITR, such as the Rep Recognition Sequence (RRS), and may be derived from adeno-associated virus (AAV). In some embodiments, the RRS may comprise SEQ ID NO: 1.

### BRIEF DESCRIPTION OF THE DRAWINGS

FIG. 1 shows the DNA sequence and a secondary structure of the AAV-2 ITR. The Rep Recognition Sequence (RRS) and the terminal resolution site (trs) are boxed. Also noted are regions A, A′, B, B′ C, C′ and D.

### DETAILED DESCRIPTION OF THE INVENTION

Knowledge of the interactions between viruses and regulatory factors is important to the development and efficient utilization of viral vectors for gene delivery. Further genetic, biochemical and functional analysis of these factors, in particular cellular factors, that modulate the unique life-cycle of adeno-associated virus (AAV) using the assays described below will broaden the understanding of this virus and facilitate the application of AAV-based vector systems. In particular, characterization of ITR-binding proteins and other molecules can provide new insights into the AAV life-cycle, including regulation of gene expression and integration, and suggest improvements for its application in therapy.

Embodiments of the present invention relate to a genetic screen that allows identification of cellular proteins and other molecules that bind to an Inverted Terminal Repeat region (ITR) or regions thereof, which includes the Rep Recognition Sequence (RRS). Embodiments of the present invention also provide other methods for identifying proteins and other molecules that bind to, or otherwise directly interact with an ITR or regions thereof. A domain sequence may be present in a single copy. Alternatively, multiple copies of the domain sequence may be present, or the domain sequences may be present in tandem. In one embodiment the sub-domain is the RRS sequence that may comprise SEQ ID NO: 1, or may be at least 90% homologous to SEQ ID NO: 1. Alternatively, the domain sequences comprises regions A, A′, B, B′, C, C′ or D or may be at least 90% homologous to the sequence of these regions.

The proteins and molecules described herein include endogenous cellular components which interact with the ITR or regions thereof in vivo and which, therefore, provide new targets for regulation of AAV or rAAV production, as well as recombinant, synthetic and otherwise exogenous compounds or molecules which may have ITR binding capacity and, therefore, may be candidates for viral regulation. Thus, in one series of embodiments, a cDNA library from any source including vertebrate cells, mammalian cells, human cells, rodent cells, invertebrate cells, insect cells, and the like cells, may be screened. In addition, cell lysates or tissue homogenates (e.g., human homogenates, lymphocyte lysates) may be screened for proteins or other compounds which bind to ITR or regions thereof. Additionally, any of a variety of exogenous compounds (e.g., non-viral), both naturally occurring and/or synthetic (e.g., libraries of small molecules or peptides), may be screened for binding capacity to viral ITRs or regions thereof. In each of these embodiments, an assay is conducted to detect binding between an “ITR component” and some other moiety. The ITR component in these assays may be a polynucleotide derived from a normal ITR.

In describing the present invention, the following terms will be employed, and are intended to be defined as indicated below.

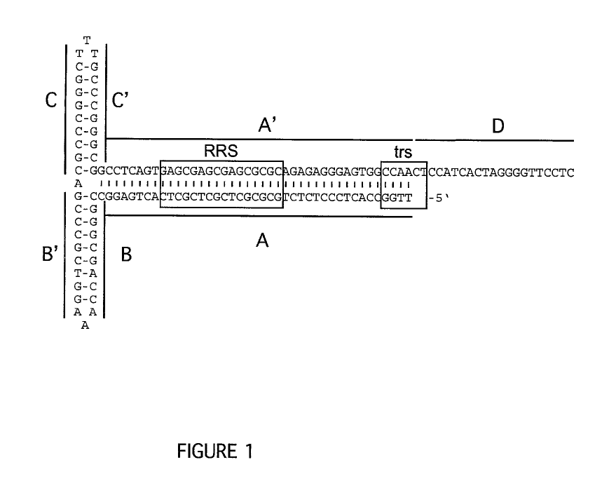

An inverted terminal repeat (ITR) is a unit of DNA that is typically about 145 nucleotides in length consisting of seven regions; A, A′, B, B′, C, C′and D (See FIG. 1). Certain regions can base pair with one another to form double-stranded regions referred to as stem structures. ITR regions that base pair to form stem structures are referred to as the A stem (A and A′), B stem (B and B′) and C stem (C and C′). One region of about twenty nucleotides (D) remains single-stranded, i.e., it does not base pair. Regions when utilized in the current invention, except for the D region (that remains single-stranded) are typically used as double-stranded nucleic acids, A and A′, B and B′and C and C′, and are typically referred to as region A, B, and C designating 5′ to 3′ regions. ITRs may be involved in several functions, such as origins of DNA replication and as viral genome packaging signals.

As used herein, “cellular” is meant that which is derived from a biological cell or present inside of a cell. The cell may be eukaryotic as in yeast or mammalian cells, or the cell may be prokaryotic as in bacterial cells. A “cellular protein” may be a protein that is typically inside of a cell, currently inside of a cell, or derived from a cell.

A “coding sequence” or a sequence which “encodes” a particular polypeptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected gene is capable of being replicated, transcribed and translated in an appropriate recipient cell.

“Viral regulatory element” refers to a cis-regulatory element within the viral genome that regulates in whole or in part various viral functions, e.g., replication, integration, etc. or controls in whole or in part the expression of sequences encoding viral products.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof Thus, for example, intervening untranslated yet transcribed sequences can be present between a promoter sequence and the coding sequence and the promoter sequence can still be considered “operably linked” to the coding sequence.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream”, “downstream”, “3′”, or “5′” relative to another sequence, it is to be understood that it is the position of the sequences in the “sense” or “coding” strand of a DNA molecule that is being referred to as is conventional in the art. “Homology” refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-strand specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules, as determined using the methods above.

A “functional homologue,” or a “functional equivalent” of a given polypeptide includes molecules derived from the native polypeptide sequence, as well as recombinantly produced or chemically synthesized polypeptides which function in a manner similar to the reference molecule to achieve a desired result. Thus, a functional homologue of AAV Rep 78 or Rep 68 encompasses derivatives and analogues of those polypeptides--including any single or multiple amino acid additions, substitutions and/or deletions occurring internally or at the amino or carboxy termini thereof, so long as replication activity remains.

By “vector” is meant any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors.

By an “AAV vector” is meant a vector derived from an adeno-associated virus serotype, including without limitation, AAV-1, AAV-2, AAV-3, AAV-4, AAV-5, AAV-6 etc. AAV vectors can have one or more of the AAV wild-type genes deleted in whole or part, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITR need not be the wild-type nucleotide sequences, and may be altered, e.g., by the insertion, deletion or substitution of nucleotides, so long as the sequences provide for functional rescue, replication and packaging. AAV vectors can be constructed using recombinant techniques that are known in the art to include one or more heterologous nucleotide sequences flanked on both ends (5′ and 3′) with functional AAV ITRs. In the practice of the invention, an AAV vector can include at least one AAV ITR and a suitable promoter sequence positioned upstream of the heterologous nucleotide sequence and at least one AAV ITR positioned downstream of the heterologous sequence. The 5′ and 3′ ITRs need not necessarily be identical or derived from the same AAV isolate, so long as they function as intended.

By “adeno-associated virus inverted terminal repeats” or “AAV ITRs” is meant the art recognized palindromic regions found at each end of the AAV genome which function together in cis as origins of DNA replication and as packaging signals for the virus. AAV ITRS, together with the AAV rep coding region, provide for the efficient excision and rescue from, and integration of a nucleotide sequence interposed between two flanking ITRs into a mammalian cell genome.

As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, etc. Furthermore, 5′ and 3′ ITRs which flank a selected nucleotide sequence in an AAV vector need not necessarily be identical or derived from the same AAV serotype or isolate, so long as they are functional. “AAV functions” refer to AAV-derived coding sequences which can be expressed to provide AAV gene products that, in turn, can function in trans for productive AAV replication. Thus, AAV functions include one, or both of the major AAV open reading frames (ORFs)—rep and cap. The Rep expression products have been shown to possess many functions, including recognition, binding and nicking of the AAV origin of DNA replication; DNA helicase I activity; and modulation of transcription from AAV (or other heterologous) promoters. The Cap expression products form the capsid necessary for packaging. AAV functions are used herein to complement AAV functions in trans that are missing from AAV vectors.

The term “helper viral functions” refers to the provision of factors that are necessary during various aspects of the AAV life cycle. AAV requires such helper functions from an unrelated helper virus (e.g., an adenovirus, a herpes virus or a vaccinia virus), in order for a productive AAV infection to occur. Particularly, it has been demonstrated that adenovirus supplies factors required for AAV promoter expression, AAV messenger RNA stability and AAV translation and replication. See, e.g., Muzyczka, N. (1992) Curr. Topics. Microbiol. and Immun. 158:97-129. In the absence of such functions, AAV establishes a latent state by insertion of its genome into a host cell chromosome. Production of viral helper functions rescues the integrated copy which can then replicate to produce infectious viral progeny. Viral helper functions can be provided by infection of a cell with a suitable helper virus or transfection of a helper plasmid (Xiao et al., 1998, supra).

AAV packaging constructs can be in the form of a plasmid, phage, transposon, cosmid, virus, or virion. The term “AAV packaging construct” refers generally to a nucleic acid molecule that includes nucleotide sequences providing AAV functions deleted from an AAV vector which is to be used to produce a transducing vector for delivery of a nucleotide sequence of interest. AAV packaging constructs are commonly used to provide transient expression of AAV rep and/or cap genes to complement missing AAV functions that are necessary for lytic AAV replication; however, helper constructs lack AAV ITRs and can neither replicate nor package themselves. A number of AAV helper constructs have been described, such as the commonly used plasmids pXX2, pAAV/Ad and pIM29+45 which encode both Rep and Cap expression products. See, e.g., Samulski et al. (1989) J. Virol. 63:3822-3828; and McCarty et al. (1991) J. Virol. 65:2936-2945, Xiao et al, (1998) supra.. A number of other vectors have been described which encode Rep and/or Cap expression products. See, e.g., U.S. Pat. No. 5,139,941.

An “AAV p5 promoter region” encompasses both promoter sequences with identity to a p5 promoter region isolated from an AAV serotype, including without limitation, AAV-1, AAV2, AAV-3, AAV-4, AAV-5, AAV-6, etc., as well as those which are substantially homologous and functionally equivalent thereto (as defined below). The AAV p5 promoter directs the expression of the long forms of Rep, and has been described and characterized. See, e.g., Lusby et al. (1982) J. Virol. 41:518-526; Laughlin et al. (1979) Proc. Natl. Acad. Sci. USA 76:5567-5571; Green et al. (1980a) J. Virol. 36:79-92; Green et al. (1980b) Cell 1:23 1-242. For purposes of defining the present invention, in the wild type AAV genome, the AAV p5 promoter region is “in its natural position” when it is bound at the 5′-terminus of the transcriptional start site of the rep coding sequence and the rep transcriptional start site is approximately 25 base pairs (bps) downstream (3′-direction) from the p5 TATA box, such that the rep ATG is approximately 60 base pairs downstream (3′ direction) from the p5 TATA box. The wild type AAV p5 promoter extends upstream (5′-direction) to include the minimum number of bases or elements necessary to initiate transcription of the long forms of Rep at levels detectable above background.

By an “AAV coding region” is meant a nucleic acid molecule which includes the two major AAV open reading frames corresponding to the AAV rep and cap coding regions (e.g., a nucleic acid molecule comprising a nucleotide sequence substantially homologous to base pairs 3 1 0 through 4,440 of the wild-type AAV genome). See, e.g., Srivastava et al. (1983) J. Virol. 45:555-564; Hermonat et al. (1984) J. Virol. 51:329-339; and Tratschin et al. (1984) J. Virol. 51:61 I619.

There are at least two types of screens used to identify cellular proteins that play a role in the AAV life-cycle. The first are genetic screens, such as those performed in yeast, to identify molecules, e.g., cellular proteins, that bind directly to DNA sequences within the viral inverted terminal repeat (ITR). Examples of this type of genetic screen can be found in EXAMPLE 2. The second approach uses biochemical techniques to isolate relevant proteins or other molecules

In one embodiment a yeast-based system for isolating genes that encode human proteins able to bind DNA sequences within the viral ITR, such as the specific region of 16 nucleotides from the ITR that is recognized by the viral Rep protein. The assay is based on the principle of the yeast one-hybrid system whereby DNA binding proteins are isolated through activation of reporter genes. This assay can be used to screen a library of human cDNAs. Once identified, the protein can be confirmed by using, for example, an in vitro assay (the electrophoretic mobility shift assay). Generation of the yeast reporter strains has been described (Cathomen et al., (2001) Proc. Natl. Acad. Sci. 98: 14991-14996). This assay system and screening approach could be adapted to other parts of the viral ITR, in order to identify other proteins that recognize specific elements (cis-regulatory elements) within the viral genome.

Binding may be detected by indirect methods (e.g., AAV production, 2D gel electrophoresis, differential hybridization, and immunoprecipitation) or by direct measures such as electrophoretic mobility shift assay (EMSA), the Biomolecular Interaction Assay (BIAcore) or alteration of gel electrophoresis (reviewed in Nowak (1995) Science 270:368-371 and Kahn (1995) Science 270:369-370). The preferred methods involve variations on the following techniques: (1) direct extraction by affinity chromatography; (2) co-isolation of ITR components and bound proteins or other compounds by immunoprecipitation; (3) BIAcore analysis; and (4) the one or two-hybrid systems (Vidal & Legrain Nucleic Acids Res. (1999) 27(4):919-29).

Other ITR-interaction assays take advantage of techniques in molecular biology that are employed to discover molecule:molecule (e.g., protein:DNA) interactions, including assays that can be can be adapted to identify binding partners (e.g., the two-hybrid systems (Field & Song, Nature 340:245-246 (1989); Chien et al., Proc. Natl. Acad Sci. USA 88:9578-9582 (1991); and Young K H, Biol. Reprod. 58:302-311 (1998); reverse two-hybrid system (Leanna & Hannink, Nucl Acid Res. 24:3341-3347 (1996); repressed transactivator system (Sadowski et al., U.S. Pat. No. 5,885,779); phage display (Lowman H B, Annu. Rev. Biophys. Biomol. Struct. 26:401-424 (1997), herein incorporated by reference); and GST/HIS pull down assays, mutant operators (Granger et al., WO 98/01879) and the like (See also Mathis G., Clin. Chem. 41:139-147 (1995); Lam K. S. Anticancer Drug Res., 12:145-167 (1997); Phizicky et al., Microbiol Rev. 59:94-123 (I 995), Drug Discovery and Evaluation: Pharmacological Assays, Vogel & Vogel (ed.) Springer Verlag (I 997); Advances in Drug Discovery Techniques, Harvey, A L (ed.) John Wiley & Sons (1998); and Biopharmaceutical Drug Design and Development, Wu-Pong et al., (ed.) Humana Press (1999)). All references herein expressly incorporated by reference.

As will be obvious to one of ordinary skill in the art, there are numerous other methods of screening individual proteins or other compounds, as well as large libraries of proteins or other compounds (e.g., phage display libraries and cloning systems from Stratagene, La Jolla, Calif.) to identify molecules that bind to ITR components. All of these methods comprise the step of mixing ITR or a fragment thereof with a test compound, allowing for binding, and assaying for bound complexes.

### Identifying DNA Interactions

Embodiments of the invention provide a method for identifying a protein or other molecule which can bind to a viral regulatory region. The method includes incubating the viral regulatory element or a recombinant cell harboring the viral regulatory element with solutions containing test proteins of interest under conditions sufficient to allow the components to interact, and measuring the binding of the protein of interest by expression of the reporter gene.

In one embodiment, the invention provides a method for screening polypeptides which bind to viral regulatory regions, thus modifying gene expression. The method includes incubating the viral regulatory region with either the proteins of interest, cell lysates, solutions or mixtures containing test proteins of interest, and the like under conditions sufficient to allow the components to interact and determining the effect of a given test protein on the expression of the reporter gene. The effect of the protein on the protein-viral regulatory DNA interaction can be measured by a number of assays, in addition to the measurement by reporter gene expression. Not only proteins, but also other molecules and compounds including peptides, polypeptides, pepidomimetics, nucleic acids, chemical compounds and biological agents may also be used in the assay to identify binders to viral regulatory elements.

### Binding Conditions

Suitable binding conditions may depend on many factors, such as the concentration of the viral regulatory element, the concentration of the binding molecule, the presence and concentration of other assay components, pH of the system, temperature, and salt concentration. The term “binding conditions” as used herein refers to the conditions that are used for the assay. The choice of whether the assay is performed in a cell-based assay or in an in vitro (cell free) system will be likely to affect the optimal binding conditions. The assay may be performed, for example, in a solution using a simple buffer, a complex biological fluid (e.g. blood, serum, urine, saliva, and many others), or a cell. The selection of conditions desirable for the preferred level of binding specificity to test for activation of the viral regulatory element can be determined by one of ordinary skill in the art. Examples of suitable binding conditions are shown in EXAMPLES 2 and 3. Typical binding conditions for a cell-based system are exemplified in EXAMPLE 2. Suitable in vitro binding conditions are exemplified in EXAMPLE 3.

### Assay Kit

The present invention may be in the form of a kit. The kit can be used for therapeutic treatment and/or for diagnosis. It is provided that the kit will be used with humans and/or animals. Further, the kit can also be used in either in vivo or in vitro systems.

### Detection, Purification, and Characterization of the Cellular Proteins and Other Molecules that Bind to the Viral Regulatory Element

In some embodiments of the invention, the detection of the protein or other molecule binding to the viral regulatory region is performed by the expression of a suitable reporter gene, described below. Other means of detection of whether the cellular proteins or other molecules are bound to the DNA may be used. Additionally, the isolation, purification, and further characterization of the detected proteins and other molecules can be performed by various biochemical techniques. Among such methods include one-dimensional and two-dimensional gel electrophoresis, electrophoretic mobility shift assays, immunoprecipitation, surface plasmon resonance-based technology (e.g., BIAcore, Pharmacia, PeaPack, N.J.) to examine DNA-protein interactions, affinity chromatography, as well as one and two hybrid assay systems.

Several methods to isolate and purify interacting proteins and other molecules of interest are known in the art. Among these are immunoprecipitation techniques, affinity chromatography techniques, or conventional column purification techniques such as size exclusion chromatography, ion exchange chromatography, HPLC, and FPLC. Immunoprecipitation methods may be used to isolate the molecule of interest either before or after the DNA interaction steps. Typically, an antibody that recognizes the protein or other molecule to be isolated is attached to a solid support, while a complex mixture is passed through. The molecule of interest will bind to the antibody. The antibody-molecule mixture is washed, and the molecule of interest is eluted. The protein or molecule of interest may be purified by affinity chromatography methods. Affinity chromatography is used to purify a molecule from a solution. For example, the viral regulatory element DNA may be attached to a solid support, such as agarose beads on a column, while the interacting protein (often in a complex mixture) or molecule is able to bind to the first member when passed through the solid support. After washing steps, the relatively purified protein or molecule is eluted.

The viral regulatory DNA sequence bound to a protein or molecule of interest can be isolated or purified by immobilizing or precipitating the complex. For example, affinity chromatography may be used to isolate DNA-protein complexes found by the use of this invention. Once the viral regulatory region-cellular protein complex has been isolated, the cellular component can be isolated and characterized by means well known in the art. For example, the cellular protein can be sequenced using methodology well known in the art. For example, the lysate may be prepared from the expression host and the lysate purified using HPLC, exclusion chromatography, gel electrophoresis, affinity chromatography, or other purification techniques.

In some embodiments, two-hybrid systems may be used to detect proteins or other molecules of interest. The two hybrid system is a yeast-based genetic assay to detect protein interactions in vivo. This assay has many useful features compared to the in vitro methods. Most notably, because the two-hybrid assay is performed in vivo, the proteins involved are more likely to be in their native conformations. In addition, purified target proteins or antibodies to the desired protein are not required. The two-hybrid assay is also a sensitive method for detecting weak and transient interactions. The two-hybrid assay is based on the fact that many eukaryotic transcriptional activators (like the yeast transcriptional activator GAL4) consist of two physically separable modular domains: one acts as the DNA-binding domain, while the other functions as the transcriptional activation domain. The DNA-binding domain localizes the transcription factor to specific DNA sequences present in the upstream region of genes that are regulated by this factor, while the activation domain (AD) contacts other components of the transcription machinery required to initiate transcription. Both domains are required for normal activation function, and normally the two domains are part of the same protein. However, it has been shown that a functional activator can be assembled in vivo from separate domains of the same or unrelated transcription factors via recombinant DNA technology.

Another method of detecting and characterizing binding proteins of interest may involve such biochemical techniques as electrophoretic separation of proteins using, for example, polyacrylamide gel electrophoresis (SDS-PAGE). Both one and two-dimensional electrophoresis methods may be suitable for detecting proteins of interest.

Yet another method of detecting and further characterizing the binding proteins or other molecules of interest is by the use of electrophoretic mobility shift assays (EMSA), (also termed gel shift assay, gel retardation assay, gel mobility assay). In this method, the nucleic acid (typically radiolabelled) is incubated with the cellular protein or other molecule of interest, then separated electrophoretically on an agarose or polyacrylamide gel. The distribution of radioactivity is then examined to see whether a given protein affects the mobility of the nucleic acid.

### Interactions Between the Viral Regulatory Region and Other Molecules

In addition to protein binding, other molecules may bind to the viral regulatory region to modulate or otherwise affect expression of the reporter gene. The term “modulation” or “modulated” as used herein refers to any change in functional activity such as activation, enhancement, increasing, interference with or suppression of the viral regulatory region, or an increase or decrease in the amount of expressed reporter gene. A “modulatory molecule” can modulate the activity of the viral regulatory region in many ways. For example, a modulator molecule may act on a viral regulatory region by affecting its conformation, folding, (or other physical characteristics), binding to other moieties (such as ligands), or activity (or other functional characteristics). Any method of modifying the activity of the viral regulatory region is suitable for the present invention, as long as the modification of activity when compared to the absence of the modulatory molecule can be assessed.

Several types of molecules may be found to bind to the viral regulatory element. As used herein, the term “molecule” includes but is not limited to proteins, enzymes, antibodies, antigens, metabolites, drugs, small molecules, nucleic acids (e.g. natural or synthetic DNA, RNA, cDNA, mRNA, tRNA, etc.), lectins, sugars, glycoproteins, salts, lipids, receptors (with or without their ligands), drug candidates, growth factors, cytokines, natural products, vitamins, gases (e.g. oxygen, CO₂, and the like), fluids, metabolites, cells, whole virus particles, and other ligands. Any molecule may be useful for binding to the viral regulatory element, whether it be a macromolecule, a small molecule, an inorganic molecule, an organic molecule, or other types of molecules. The molecules may be naturally occurring, such as the natural products made by plants or animals, or the molecules may be synthetic. Functional or structural analogues or mimics of such compounds that exhibit substantially the same binding activity are also included within the meaning of the term as used herein. The type, size or shape of the molecule is not important so long as the molecules can bind to the viral regulatory element.

Such a molecule may be added exogenously, and may also be derived from a chemical library. For example, combinatorial libraries of natural or synthetic molecules may be used. The term “chemical library” or “array” refers to an intentionally created collection of differing molecules which can be prepared synthetically and screened for biological activity in a variety of different formats (e.g., libraries of soluble molecules, libraries of molecules bound to a solid support).

### The Yeast One-Hybrid Screen

The yeast one-hybrid assay is a genetic screen for isolating genes that encode proteins with a specific DNA-binding activity. In 1993, Wang & Reed first used the one-hybrid assay to clone the gene encoding the transcription factor OLF-1. The one-hybrid system offers high sensitivity because detection of the DNA-protein interaction occurs in vivo while proteins are in their native configuration. In addition, the gene encoding the DNA-binding protein of interest is immediately available after the library screening. The screen is based on the finding that many eukaryotic transcription factors are composed of physically and functionally independent domains: a DNA-binding domain and a transcriptional activation domain. Thus, fusion proteins can be constructed that activate transcription through binding to a target sequence. Provided that such a protein exists, any DNA target element can be used to trap a cellular protein with a DNA-binding domain specific for that element.

The yeast one-hybrid system is typically used to identify genes encoding proteins that bind to a nucleic acid target, such as a cis-acting regulatory element or any other short DNA-binding sequence. Detection of the DNA-protein interactions occurs while proteins are in their native configuration, and the gene encoding the DNA-binding protein of interest is available immediately after library screening (See, for example, Sieweke (2000) “Detection of transcription factor partners with a yeast one hybrid screen” Methods Mol Biol; 130:59-77). A general explanation of the yeast one-hybrid system can be found, for example, in U.S. Pat. No. 6,046,165, hereby incorporated by reference in its entirety.

The yeast one-hybrid system can be used to assay the binding between viral regulatory regions and their binding proteins in vivo. After incubating or contacting the viral regulatory region with cellular proteins, the assay measures activation of a reporter gene in response to protein binding to a site positioned upstream of a basal promoter. The principle of the yeast one-hybrid system can be adapted for use in other types of cells, including bacteria and mammalian cultured cells. “Incubating” as used herein includes conditions which allow contact between the test protein or other compound and the viral regulatory DNA region. “Contacting” includes in solution and solid phase. The test protein may optionally be derived from a library of polypeptides. Polypeptides identified in the screening method of the invention can be further evaluated, detected, cloned, sequenced, and the like, either in solution or after binding to a solid support, by any method usually applied to the detection of a polypeptide sequence.

A variety of other agents may be included in the screening assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents, etc., may be used. The mixture of components are added in any order that provides for the requisite binding. Incubations are performed at any suitable temperature, typically between 4° C. and 40° C. Incubation periods are selected for optimum activity, but may also be optimized to facilitate rapid high-throughput screening. Typically between 0.1 and 10 hours will be sufficient.

### Generation of the Yeast Reporter Strain

To conduct the assay, a yeast reporter strain must be made, having the sequence of a DNA target element upstream of both a nutritional selection marker and a reporter gene. Typically, the reporter gene may encode a protein that can be detected by histochemical staining (e.g., beta-galactosidase) or by autofluorescence (i.e., Green Fluorescent Protein (GFP) or variants). One of skill in the art can identify a number of other reporter genes for use in the screening method of the invention. Examples of other reporter genes of use with the invention are luciferase, chloramphenicol acetyltransferase, and beta-glucuronidase.

A protein can affect reporter gene expression by either stimulating or inhibiting the expression of the reporter gene. A protein “inhibits” reporter gene expression if the level of transcripts or protein product produced from the reporter gene is decreased as compared with the level in the absence of the test compound. A protein “stimulates” reporter gene expression if the if the level of transcripts or protein product produced from the reporter gene is increased. The effect of the protein of interest on the reporter gene transcription can be measured by assessing the expression of the reporter by methods well known in the art (e.g., Northern blots; EMSA). Alternatively, production of protein product from the reporter gene can be measured by methods well known in the art (e.g., ELISA or RIA; Western blots; SDS-PAGE).

In some embodiments, the one-hybrid system employs a reporter gene that confers growth under conditions of starvation for a particular nutrient, usually an amino acid such as histidine. When these reporter strains are transformed with plasmids and plated onto media that lack the appropriate nutrient, only those transformants that are able to activate the nutritional reporter gene, will survive. Examples of nutritional reporter genes that may be used include the HIS3 gene, which codes for a biosynthetic enzyme necessary for the production of the amino acid histidine, the LEU2 gene, which codes for a biosynthetic enzyme necessary for the production of the amino acid leucine, TRP1, which codes for a biosynthetic enzyme necessary for the production of the amino acid tryptophan, LYS2 which codes for a biosynthetic enzyme necessary for the production of the amino acid Lysine, and MET15, which codes for a biosynthetic enzyme necessary for the production of the amino acid Methionine. Other nutritional reporter genes include URA3, which is involved in uracil biosynthesis, and the two genes involved in adenine biosynthesis, ADE2 and ADE8. It may also be useful to use a double reporter system by using nutrient marker genes and LacZ or another reporter gene. The use of two reporters permits a more stringent library screening. The plasmids are then used to generate the yeast reporter strain by sequentially integrating the HIS3 and LacZ reporters into the yeast genome at two different loci (his3 and ura3, respectively). Integration is straightforward because the plasmids provided by different commercial suppliers permit site-specific recombination with high frequency. By marker gene selection, yeast recombinants with genomically integrated reporters are obtained.

The reporter strain may be generated, for example, by the following method. Two sequential sets of anti-parallel oligonucleotides containing one oligonucleotide having the following sequence:

5′-AGCTTCAGTGAGCGAGCGAGCGCGCAGGT (SEQ ID NO:2),

5′-CGACCTGCGCGCTCGCTCGCTCACTGA (SEQ ID NO:3), and two copies of the RRS having the following sequence:

5′-TCGAAGTGAGCGAGCGAGCGCGCAGGTGAGCGAGCGAGCGCGCAGC(SEQ ID NO:4)

5′-TCGAGCTGCGCGCTCGCTCGCTCACCTGCGCGCTCGCTCGCTCACT (SEQ ID NO:5) (RRS underlined), are inserted into the polylinker of pLacZi (BD Clontech, Palo Alto, Calif.) to generate reporter plasmid pRRS3.LacZi. The resulting plasmid is linearized with NcoI and transformed to yeast strain YM4271 (BD Biosciences Clontech (Palo Alto, Calif.)), preferably using the LiAc transformation procedure, described below.

### Transformation of Yeast Using the LiAc Procedure

To transform yeast using the LiAc procedure, 1 ml of appropriate SD medium is inoculated with several colonies, 2-3 mm in diameter, and vortexed for 5 min to disperse any clumps. The solution is transferred into a flask containing 50 ml of appropriate SD medium, incubated at 30° C. for 1 day with shaking at 200 rpm to stationary phase (OD₆₀₀ >1.5). A sufficient portion of this culture is then diluted into a flask containing 300 ml of YPDA to bring the OD₆₀₀ to 0.2-0.3. This flask is incubated at 30° C. for 3 h with shaking at 200 rpm (OD₆₀₀ should be 0.4-0.6).

The cells are transferred into 50 ml tubes and centrifuged at 1,000×g for 5 min at room temperature. The supernatant is discarded, and each cell pellet is resuspended in 5 ml sterile TE. The cells are pooled into one tube, centrifuged at 1,000×g for 5 min at room temperature, and the supernatant is discarded. The cell pellet is resuspended in 1.5 ml of freshly prepared, sterile 1X TE/1X LiAc. 0.5 μg of plasmid DNA and 100 μg of herring testes carrier DNA is added to a fresh 1.5-ml tube and mixed (the carrier DNA should be denatured just prior to use by placing it in a boiling water bath for 20 min and immediately cooling it on ice).

For the transformation procedure, 100 μl of yeast competent cells to are added to each tube and vortexed. 600 μl of sterile PEG/LiAc solution is added to each tube and vortex at high speed for 10 sec to mix. The mixture is then incubated in a 30° C. water bath for 30 min. 70 μl of DMSO is added, mixing by gentle inversion rather than by vortexing. The cells are subjected to a heat shock for 5 min in a 42° C. water bath, then chilled on ice for 1 min. Cells are centrifuged for 5 sec at 14,000 rpm at room temperature, and the supernatant is removed. The cells are resuspended in 500 μl of appropriate SD medium, and incubated in a 30° C. water bath for 60 min.

To select for transformants, 100 μl of the above mixture is placed on an SD agar plate that will select for the desired transformants. The plates are incubated at 30° C. until colonies appear (generally 2-4 days). In particular, transformants may be plated on SD/-Ura plates and incubate for 3 days at 30° C. to select for colonies with an integrated LacZ reporter gene. Large colonies are picked and placed on a SD/-Ura plate and incubated for 2 days at 30° C.

### Use of a Colony Lift Filter Assay to Determine LacZ Background Expression

A colony lift filter assay can be used to determine LacZ background expression. The assay may be performed according to the following general method: a sterile Whatman filter is presoaked by placing it in 2.5 ml of Z buffer/X-gal solution in a clean 10 cm plate. Using forceps, a clean, dry filter is placed over the surface of the plate of colonies to be assayed. The filter is gently rubbed with the side of the forceps to help colonies cling to the filter. Holes are poked through the filter into the agar in three or more asymmetric locations to orient the filter to the agar. The wetted filter is lifted off of the agar plate with forceps and transferred (colonies facing up) to a pool of liquid nitrogen. Using the forceps, the filters are completely submerged for 10 sec. After the filter has frozen completely, it is removed from the liquid nitrogen and allowed to thaw at room temperature.

The filter, colony side up, is carefully placed on the presoaked filter, avoiding the trapping of air bubbles under or between the filters. The filters are incubated at room temperature and checked periodically for the appearance of blue colonies. The B-galactosidase producing colonies are identified by aligning the filter to the agar plate using the orientation marks. Using this method, clones producing low background amounts of β-galactosidase can be identified and used as reporter strain YM.RRS3.LacZ.

### Generation of Reporter Plasmid Containing the HIS3 Reporter Gene

To generate reporter plasmid pRRS2.HISi-1, a set of anti-parallel oligonucleotides containing two copies of the RRS,

5′-CGCGGTGAGCGAGCGAGCGCGCAGGTGAGCGAGCGAGCGCGCAGT SEQ ID NO:6)

5′-CTAGACTGCGCGCTCGCTCGCTCACCTGCGCGCTCGCTCGCTCACC (SEQ ID NO:7)

(RRS underlined), is inserted into the polylinker of plasmid pHISi-1 (BD Biosciences Clontech (Palo Alto, Calif.)) at the SacII and XbaI sites to generate reporter plasmid pRRS2.HISi-1. The plasmid pRRS2.HISi-1 is then linearized with xhoI and transformed to yeast strain YM.RRS3.LacZ using the LiAc transformation procedure described above. Transformants are grown on SD/-Ura,-His plates and incubated for 6 days at 30° C. to select for colonies with an integrated HIS3 reporter gene. Note that the RRS2.HIS3 reporter is used in two different ways. For plasmid integration, leaky HIS3 expression from pRRS2.HISi-1 permits enough colony growth on SD/-His medium (without 3-AT) to use it as a selectable marker. In the library screening, background growth due to leaky HIS3 expression is suppressed by adding 3-AT to the medium, and the RRS2.HIS3 reporter is used to detect interaction of a library protein with the target element.

Large, well-isolated colonies are restreaked on SD/-His plates and incubate for 4 days at 30° C. To test colonies with integrated pRRS2.HISi-1 for HIS3 background expression, a single colony is picked and suspended in 1 ml of TE buffer. 5 μl of the suspension is plated on SD/-His plates supplemented with 0, 15, 30, 45, and 60 mM 3-AT. The reporter strain with the lowest HIS3 background expression level is identified for use as reporter strain YM.RRS2.HIS/RRS3.LacZ (Cathomen, (2001) supra).

The reporter strain YM.RRS2.HIS/RRS3.LacZ may be validated by transformation with plasmids pADH.RepTZAD and pADH.RepTZ using the LiAc transformation procedure. The chimeric proteins RepTZ and RepTZAD contain the major DNA-binding motif of Rep fused to an oligomerization domain required for binding to the RRS. RepTZAD contains an additional transcriptional activation domain that will induce expression of the reporter genes upon binding to the RRS element. Transformants are patched on SD/-Ura,-Leu,-His supplemented with 15 mM 3-AT to analyze whether expression of RepTZAD supports growth in the absence of histidine. In parallel, transformants are patched on SD/-Ura,-Leu supplemented with X-gal to control for the specific activation of the LacZ reporter.

The In vivo LacZ Plate Assay Using X-gal may be performed by pouring SD/-Ura,-Leu agar plates containing X-gal (80 mg/L) and 1×BU salts. It is important to note that X-gal is heat-labile and will be destroyed if added to medium >55° C. After allowing plates to dry at room temperature for 2-3 days, the transformants are patched to the plates and incubated at 30° C. for 2-4 days, checking every 12 hours for development of blue color.

### Library Screening

Among the standard procedures for isolating cDNA sequences of interest is the formation of plasmid- or phage-carrying cDNA libraries which are derived from reverse transcription of mRNA from donor cells that have a high level of genetic expression.

The cDNA library may be screened for a gene encoding a DNA-binding protein of interest, the reporter strain is transformed with a library of proteins fused to the GAL4 transcriptional activation domain (GAD). Transformants are plated on selective medium. If a specific hybrid protein interacts with the inserted target element, expression of the HIS3 reporter gene is activated, allowing colony growth on minimal medium lacking histidine. Since HIS3 reporter gene expression is leaky, selection medium is supplement with 3-amino-1,2,4-triazole (3-AT) to suppress HIS3 background expression. A 62 -galactosidase assay is performed to verify the DNA-protein interaction independently. Next, the library plasmids are isolated from positive yeast clones and amplified in E. coli. To help eliminate any false positive clones, the library plasmids are transformed into a yeast control strain, which can be generated by integrating a LacZ reporter without target elements.

A human cDNA library may be screened, for example, according to the procedure described below. The reporter strain YM.RRS2.HIS/RRS3.LacZ is transformed with 30 μg of a HeLa cell cDNA library in vector pGAD-GH (BD Biosciences Clontech (Palo Alto, Calif.)) using the large scale LiAc transformation procedure described above. The amount of library plasmid screened should have a complexity of more that 106 individual clones.

### Large-scale Yeast Transformation

The method described below may be used for large-scale yeast transformation. The protocol is especially useful for screening greater than 10⁶ independent clones. 1 ml of YPDA is inoculated with several 2-3 mm large colonies of the YM.RRS2.HIS/RRS3.LacZ reporter yeast strain and vortexed to disperse any clumps. The cell suspension is transferred to a flask containing 50 ml of YPDA and incubated at 30° C. overnight with shaking at 200 rpm to stationary phase (OD₆₀₀>1.5). The overnight culture is diluted into 300 ml of YPDA to produce an OD₆₀₀=0.2-0.3, then incubated at 30° C. for 3-4 h with shaking at 200 rpm until culture reaches OD₆₀₀=0.8.

The culture is centrifuged in six 50 ml tubes at 3,000×g for 5 min at room temperature, the supernatant is discarded, and the resulting pellet is resuspended in 25 ml of TE buffer. The cells are centrifuged again at 3,000×g for 5 min at room temperature, and each pellet is resuspended in 3 ml of freshly prepared, sterile 1×TE/LiAc. The tubes are mixed well by vortexing. The following is added to each tube in order: 2.4 ml of 50% PEG, 360 μl of 1 M LiAc, 100 μl of herring testes carrier DNA (10 mg/ml), 5 μl of library plasmid DNA (1 μg/μl), and 735 μl H₂O. The tubes are vigorously vortexed until the cell pellet is completely resuspended, then the tubes are incubated at 30° C. for 30 min with shaking at 200 rpm. 360 μl of DMSO is added and mixed well by gentle inversion rather than vortexing. The tubes are heat shocked for 30 min in a 42° C. water bath, swirling every 5 min to mix.

The tubes are chilled on ice for 2 min, and centrifuged at 3,000×g for 5 min at room temperature. The supernatant is discarded. Each cell pellet is resuspended in 7 ml of TE buffer. 2 μl of the transformation mixture is diluted in 100 μl of TE buffer, then plated on a 10 cm SD/-Ura,-Leu plate to determine the transformation efficiency (should be >10⁶ individual clones). 400 μl of the transformation mixture is then plated on each 15 cm SD/-Ura,-His,-Leu,+15 mM 3-AT plate (90-100 plates total), spreading cells immediately after pipetting to avoid localized dilutions in the 3-AT concentration. The plates are incubated at 30° C. for 7-10 days. Growth is then assessed.

The largest colonies are patched on SD/-Ura,-Leu plates containing X-gal to perform an in vivo LacZ Plate Assay (described above). The plates are incubated for 3 days at 30° C. and score the extent of blue staining of the patched clones.

### Isolation of Plasmid DNA from Yeast Using QIAprep™ Spin Miniprep Kit from QIAGEN

Plasmids from the positive clones can be isolated using a modified QIAGEN (Valencia, Calif.) spin column DNA mini-prep procedure or other equivalent procedures. To do this, 2 ml overnight cultures are grown, highly pure mini-prep DNA is prepared using QIAGEN spin columns. A single colony is then inoculated into 5 ml of SD/-Ura,-Leu and the culture is grown overnight. The cells are pelleted by centrifugation for 5 min at 5,000×g, removing the resulting supernatant. The pellet is resuspended in 250 μl of buffer PI containing 0.1 mg/ml RNAase A and transferred to a 1.5 ml microfuige tube. 50-100 μl of acid-washed glass beads is added and vortexed for 5 min. The tube is then allowed to settle. The supernatant is transferred to a fresh 1.5 ml microfuige tube. 250 μl of buffer P2 is added, inverted 5 times to mix and incubated at room temperature for 5 min. 350 μl of buffer N3 is added and inverted 5 times to mix. The lysate is centrifuged for 10 min at 10,000×g and the cleared lysate is transferred to a QIAprep™ spin column. The column is centrifuged for 1 min at 10,000×g, discarding the flow-through. 750 μl of buffer PE is added and centrifuged for 1 min at 10,000×g, again discarding the flow-through. The column is centrifuged an additional 1 min at 10,000×g to remove residual wash buffer. The QIAprep™ spin column is placed into a clean 1.5 ml microfuige tube. 25 μl of buffer EB is added, the solution is allowed to settle for 1 min, followed by centrifugation for 1 min at 10,000×g. The typical yield of plasmid is about 1 μg, and is used for E. coli transformation, described below.

### Transformation of E. coli

For transformation of E. coli, 2-5 μl of the eluate from the above described column should yield about 30 colonies. The control strain YM.RRS0.LacZ, which was generated by integrating the empty pLacZi plasmid (BD Biosciences Clontech (Palo Alto, Calif.)) is transformed into strain YM4271 (BD Biosciences Clontech (Palo Alto, Calif.)), with isolated library plasmids. This is patched onto X-gal plates to perform an In vivo LacZ Plate Assay. This control tests that the activation is dependent upon binding to the RRS and not due to a non-specific interaction. Transformants that remain white are considered as real positives.

The library is subcloned by insertion into a mammalian expression vector providing an N-terminal epitope tag. For instance, subcloning the EcoRI—XhoI insert of a cDNA library in pGAD-GH into plasmid pCS3+MT provides an N-terminal 6×Myc epitope in-frame with the library open reading frame. Plasmid pCS3+MT contains a bacteriophage SP6 and the CMV IE94 promoter, allowing expression of the tagged protein in vitro and in cultured cells.

### Confirmation of DNA Binding

The DNA binding can be confirmed by other independent methods. Useful methods include electrophoretic mobility-shift assays (described in EXAMPLE 3).

To further analyze the binding proteins, other proteins of interest, or cell lysates, immunological methods such as immunoblot analysis of the lysates or proteins of interest may be performed using antibodies that are specific to proteins of interest. The term “antibody” as used in this invention includes intact molecules as well as fragments thereof, such as Fab, F(ab′)2, and Fv capable of binding to an epitopic determinant present in DAS5 polypeptide. Such antibody fragments retain some ability to selectively bind with its antigen or receptor. Methods of making these antibody fragments are known in the art. (See for example, Harlow and Lane, Antibodies: A Laboratory Manual, Cold Spring Harbor Laboratory, New York (1988), incorporated herein by reference). As used in this invention, the term “epitope” refers to an antigenic determinant on an antigen to which the paratope of an antibody binds. Epitopic determinants often consist of chemically active surface groupings of molecules such as amino acids or sugar side chains and usually have specific three dimensional structural characteristics, as well as specific charge characteristics.

In practicing the invention, an epitope tagging system may also be useful as an additional aid in tracking the binding activities of proteins of interest. In this method, the DNA encoding a short peptide of approximately 3-10 amino acids is fused to a cellular protein of interest. The expressed protein can then be visualized by commercially available antibodies that recognize the epitope tag. Proteins that bind to the viral regulatory region can be epitope tagged to allow for ease in characterization of the protein-DNA interaction. The method may be useful, for example, to purify such proteins by immunoprecipitation or affinity chromatography, or for ease in characterization of the binding proteins using immunoblotting and immunofluorescence microscopy.

The ITR can be used in an affinity chromatography procedure to isolate proteins that recognize the viral genome. For example, the ITR can be isolated and labeled with biotinylated nucleotides. The labeled probe can be used for DNA affinity purification of cellular proteins from a cellular nuclear extract. The isolated proteins can be identified by micro-sequencing with mass spectrometry. Biotinylated ITR have been used to isolate complexes via streptavidin beads. This approach has been used to isolate cellular proteins that bind to a viral origin of replication (Deng, Z, et al., Telomeric proteins regulate episomal maintenance of Epstein-Barr virus origin of plasmid replication. Mol Cell. 2002; 9(3):493-503).

The practice of the present invention will employ, unless otherwise indicated, conventional methods of virology, microbiology, molecular biology and recombinant DNA techniques within the skill of the art. Such techniques are explained filly in the literature. See, e.g., Sambrook, et al. Molecular Cloning: A Laboratory Manual (Current Edition); DNA Cloning: A Practical Approach, vol. I & 11 (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing descriptions, the following examples, and the accompanying drawing. Such modifications are intended to fall within the scope of the appended claims. The following examples are intended to illustrate but not limit the invention.

The invention will now be described in greater detail by reference to the following non-limiting examples.

### EXAMPLE 1

### Materials, Yeast Strains, and Solutions for the Yeast One-Hybrid Screen

### Solutions for the Assays may be Prepared According to the Following Methods

YPDA medium/agar: Dissolve 20 g/L Difco peptone (Becton Dickinson, Franklin Lakes, N.J.), 10 g/L Yeast extract, 20 g/L Agar (for plates only), 15 ml of 0.2% adenine hemisulfate in 950 ml of H₂O. Adjust the pH to 6.5 if necessary and autoclave. Allow medium to cool to 55° C., then add 50 ml of a sterile 40% dextrose (glucose).

SD/-Ura medium/agar: Dissolve 6.7 g yeast nitrogen base without amino acids and 20 g agar (for plates only) in 835 ml H₂O. Add 15 ml of 0.2% adenine hemisulfate and 100 ml of 10×DO/-Ura solution, adjust the pH to 5.8 if necessary, and autoclave. Allow medium to cool to 55° C., then add 50 ml of a sterile 40% dextrose (glucose).

SD/-Ura,-Leu,-His, +15 mM 3-AT agarplates: Omit Leucine and Histidine in the 10×DO solution and add 15 ml of a sterile 1 M 3-AT stock solution to each L of medium after it is cooled to 55° C.

10×DO/-Ura solution: Dissolve L-Adenine hemisulfate salt 200 mg/L, L-Arginine HCl 200 mg/L, L-Leucine 1000 mg/L, L-Histidine HCl monohydrate 200 mg/L, L-Isoleucine 300 mg/L, L-Lysine HCl 300 mg/L, L-Methionine 200 mg/L, L-Phenylalanine 500 mg/L, L-Threonine 2000 mg/L, L-Tryptophan 200 mg/L, L-Tyrosine 300 mg/L, L-Valine 1500 mg/L in 1 L of H₂O. Autoclave and store at room temperature.

PEG/LiAc solution (polyethylene glycol/lithium acetate): Always prepare fresh just prior to use. To prepare 10 ml of solution, mix 8 ml of sterile 50% PEG 3350 with 1 ml of 10×TE (0.1 M Tris-HCl, 10 mM EDTA, pH 7.5) and 1 ml of 1 M LiAc pH 7.5.

Z buffer/X-gal solution: 100 ml Z buffer, 0.27 ml 3-mercaptoethanol, and 1.67 ml X-gal stock solution. Z buffer: Dissolve Na₂HPO₄-7H₂O 16.1 g/L, NaH₂PO₄-H₂ O 5.50 g/L, KCl 0.75 g/L, and MgSO₄-7H₂O 0.246 g/L, adjust to pH 7.0, autoclave and store at room temperature for up to 1 year. X-gal stock solution: Dissolve 5-bromo-4-chloro-3-indolyl-β-D-galactopyranoside (X-gal) in N,N-dimethylformamide (DMF) at a concentration of 20 mg/ml and store in the dark at −20° C. 10X BU salt solution: Dissolving 70 g Na₂HPO₄-7H₂O and 30 g NaH₂PO₄ in 1 L of H₂O. Adjust to pH 7.0, autoclave and store at room temperature.

Other materials that may be required to perform the one-hybrid Screen may be prepared as follows: Incubator (set at 30° C.); vertical platform shaker (at 30° C.); Peptone, yeast extract, Agar, yeast nitrogen base without amino acids (Difco, distributed by BD Diagnostic Systems, Sparks, Md.); KCl, MgSO₄-7H₂O, Na₂HPO₄-7H₂O, NaH₂PO₄-H₂ O, lithium acetate, dextrose, amino acids, polyethylene glycol (PEG) 3350, 5-bromo-4-chloro-3-indolyl-β-D-galactopyranoside (X-gal), N,N-dimethylformamide (DMF), 3-aminotriazol (3-AT), β-mercaptoethanol (Sigma, St. Louis, Mo.); Herring testes carrier DNA (10 mg/ml) (BD Clontech, Palo Alto, Calif.).

Plasmids useful for the invention include: Plasmids pLacZi and pHISi-1 (BD Clontech, Palo Alto, Calif.); Plasmids pCS3+MT, pADH.Rep.TZ.AD and pADH.Rep.TZ (described herein); HeLa cell cDNA library in vector pGAD-GH (BD Clontech, Palo Alto, Calif.); Yeast strain YM4271 (BD Clontech, Palo Alto, Calif.); Yeast strains YM.RRS0.LacZ, YM.RRS3.LacZ, and YM.RRS2.HIS/RRS3.LacZ,(described herein).

### EXAMPLE 2

### Yeast One-Hybrid Screen

A genetic screen based on the yeast one-hybrid assay (Wang, M. M. & Reed, R. R. (1993) Nature (London) 364, 121-126 was designed to identify cellular proteins that bind the RRS in vivo. We recently generated chimeric Rep proteins fused to a transcriptional activation domain (Cathomen et al. (2000) supra) that activate expression of an integrated LacZ reporter gene through binding to RRS motifs upstream of a minimal promoter in Saccharomyces cerevisiae strain YM.RRS3.LacZ. To screen for cellular RRS-binding proteins, an RRS-dependent HIS3 expression cassette was integrated into the mutant his locus to generate reporter strain YM.RRS2.HIS/RRS3.LacZ. The strain was validated by using hybrid proteins RepTZ and RepTZAD. Both proteins contain the major DNA-binding motif of Rep fused to an oligomerization domain required for binding (Cathomen et al. (2000) supra). RepTZAD contains an additional transcriptional activation domain. All transformants grew on nonselective plates (YPDA). Hybrid proteins that bind the RRS and activate the HIS3 cassette allowed growth in the absence of histidine and activated the LacZ gene to give rise to blue colonies on X-gal plates.

A genetic screen identifies cellular proteins that bind the RRS motif. Reporter strain YM.RRS2.HIS/RRS3.LacZ contains integrated HIS3 and LacZ reporter cassettes driven from minimal yeast promoters with two or three upstream tandem copies of the RRS. Control strains YM.RRS3.LacZ and YM.RRS0.LacZ contain a LacZ cassette with three upstream tandem copies or no RRS. Protein RepTZ comprises residues 1-244 of Rep, a modified leucine zipper (RepTZ), a nuclear localization signal (NLS), and a Myc epitope tag. RepTZAD additionally contains the transcriptional activation domain (AD) of VP16.

Yeast in vivo plate assays demonstrate RRS binding. Strains expressing RepTZAD or RepTZ served as positive and negative controls, and clone A25 was isolated in the one-hybrid screen. Transformed yeast cells were grown on nonselective medium (YPDA) and on selection medium [SD/Ura,Leu,His, 15 mM 3-amino-1,2,4-triazole (3-AT)]. Interaction was confirmed on plates supplemented with X-gal to detect β-galactosidase activity. Specificity of the DNA-binding activity was confirmed in strains YM.RRS3.LacZ and YM.RRS0.LacZ. (D) EMSA identifies ITR-binding proteins. Positive clones were translated in vitro in the presence of [³⁵S]methionine and separated on a 12% SDS-polyacrylamide gel.

The reporter strain was transformed with a human cDNA library fused to a transcriptional activation domain. A total of 3×10⁶ transformants were screened by selection on plates lacking histidine. Activation of LacZ was tested on X-gal plates, and library plasmids were rescued from the 100 most positive clones. Strain YM.RRS3.LacZ contains three RRS elements upstream of a minimal promoter driving β-galactosidase expression (Cathomen et al. (2000) supra) and was used to reconfirm positive interactions independent of HIS3 growth selection. Strain YM.RRS0.LacZ does not contain an RRS motif and was used to exclude false positives.

The cDNAs isolated from the screen were subcloned into expression vector pCS3+MT. Proteins were translated in vitro, analyzed by SDS/PAGE and tested for their ability to bind a ³²P-labeled ITR probe by EMSA. Four of 55 proteins were analyzed. Clone A25 interacted with the ITR specifically and contained an ORF corresponding to residues 308-449 of the human ZF5.

The method described below is a more detailed example of the above-described yeast one-hybrid screen. All yeast manipulations were performed as described in the manufacturer's user manuals(BD Biosciences Clontech (Palo Alto, Calif.))). The one-hybrid screen used yeast strains containing integrated marker genes under the control of a minimal yeast promoter and upstream RRS elements. A set of antiparallel oligonucleotides containing two copies of the RRS were cloned into the polylinker of plasmid pHISi-1 (BD Biosciences Clontech (Palo Alto, Calif.)). The resulting plasmid was linearized with NcoI and integrated into the mutant his locus of strain YM.RRS3.LacZ (Cathomen, T. et al. (2000) J Virol. 74, 2372-2382) to generate YM.RRS2.HIS/RRS3.LacZ. For the one-hybrid screen, YM.RRS2.HIS/RRS3.LacZ was transformed with 60 μg of a HeLa cell cDNA library in vector pGAD-GH (BD Biosciences Clontech (Palo Alto, Calif.))), allowing expression of the cDNA as a chimeric protein fused to the GAL4 activation domain. A total of 3×10⁶ transformants were screened by selection on synthetic dropout (SD) medium minus uracil, leucine, and histidine (SD/Ura,Leu,His) plates supplemented with 15 mM 3-amino-1,2,4-triazole (Sigma, St. Louis, Mo.) to suppress leaky HIS3 expression. After seven days, large colonies were picked and patched on SD/Ura,Leu plates containing 5-bromo-4-chloro-3-indolyl-β-D-galactopyranoside (X-gal, Sigma, St. Louis, Mo.). Plates were incubated for three days at 30° C. and assessed for blue colonies. Expression plasmids were isolated from positive yeast clones, amplified in Escherichia coli (DH5), and transformed into control strains YM.RRS3.LacZ (Cathomen et al. (2000) supra) and YM.RRS0.LacZ, which were generated by integrating the empty pLacZi plasmid (BD Biosciences Clontech (Palo Alto, Calif.)) into strain YM4271 (BD Biosciences Clontech (Palo Alto, Calif.)). Transformants were patched onto X-gal plates, and hybrid proteins that induced blue staining in YM.RRS3.LacZ but not in YM.RRS0.LacZ were analyzed further. The library inserts were subcloned in-frame with an upstream epitope tag (6×Myc) in expression vector pCS3+MT (courtesy of T. Hunter, The Salk Institute) by digestion with EcoRI and XhoI. Plasmid pCS3+MT contains the bacteriophage SP6 and cytomegalovirus (CMV) IE94 promoters, allowing expression in vitro and in vivo.

Plasmids. Sequence analysis revealed that clone A25 encodes a C-terminal fragment of transcription factor ZF5 (residues 308-449). The full-length ZF5 cDNA was amplified from a HeLa cDNA library by PCR and subcloned into pCS3+MT. Site-directed mutagenesis (QuikChange, Stratagene, La Jolla, Calif.) at codon positions 334/335 (AGCTGT-ACTAGT) and 362/363 (GCGTGC-GCTAGC) led to cysteine-to-serine replacements in zinc fingers 3 (ZF5 3) and 4 (ZF5 4). Subcloning into vector pRK5 (Cathomen et al. (2000) supra) generated plasmids pRK5.ZF5C, pRK5.ZF5, pRK5.ZF53, and pRK5.ZF54. Reporter plasmid pGL2p.5.Luc contains nucleotides 190-320 of the AAV2 genome cloned into pGL2-Basic (Promega; Madison, Wis.). Plasmids pcDNA.Rep78, pcDNA.RepTZAD, pcDNA.RepTZ, pGL3.ITR/p5.Luc, pGL3.ITR/M1.Luc, pNTC244, and pAAV.GFP have been described (Cathomen et al. (2000) supra; Chejanovsky, N. & Carter, B. J. (1990) J Virol. 64, 1764-1770; and Grifinan, M. et al. (1999) J Virol. 73, 10010-10019).

In Vitro Translation and Electrophoretic Mobility-Shift Assays (EMSAs). Library clones in vector pCS3+MT were in vitro translated in the absence or presence of Tran-³⁵S-label (ICN, Costa Mesa, Calif.) by using the SP6 TNT coupled reticulocyte lysate system (Promega, Madison Wis.). The ITR probe was prepared and the EMSA was performed as described (Weitzman, M. D. et al (1994) Proc. Natl. Acad. Sci. USA 91,5808-5812; Cathomen et al. (2000) supra,). The 32-base pair-long double-stranded RRS oligonucleotide probe contains the RRS motif of the ITR. The core sequences for the wild-type and mutant probes are 5′CTGCGC(GCTC)3AC and 5′-CTCCGC(CCTC)3AC, respectively (RRS motifs are in italics). For supershift analysis, 1 μl of anti-Myc antibody (1:5 dilution, Invitrogen, Carlsbad, Calif.) was included, and in competition experiments a 1-, 5-, or 25-fold molar excess of unlabeled oligonucleotide substrate was added to the binding reaction.

### EXAMPLE 3

### Human Zf5 Binds to RRS Mitifs In vitro

A full-length cDNA of ZF5 was prepared by PCR, and zinc finger mutants were generated by site-directed mutagenesis. Because zinc finger 3 or 4 was suggested to be critical for DNA binding (Obata, T. et al. (1999) Biochem. Biophys. Res. Commun. 255, 528-534), the first cysteine of the respective zinc fingers was changed to a serine (C335S and C363S), giving rise to ZF5 3 and ZF5 4. Proteins were translated in the presence of [³⁵S]methionine, and expression was confirmed by SDS/PAGE.

Binding of proteins was assessed in vitro by EMSA using a ³²P-labeled oligonucleotide probe containing a single RRS motif (Cathomen et al. (2000) supra). ZF5 binding to the RRS in vitro is sequence-specific. ZF5 consists of a POZ domain, a stretch of acidic residues (Ac), and five C₂H₂-type zinc fingers. Mutations in ZF5 3 and ZF5 4 are shown below. The C-terminal fragment of ZF5 isolated in the screen (ZF5C) contains residues 308-449. Proteins were tagged with an N-terminal Myc epitope (6Myc). Proteins were synthesized in the presence of [³⁵S]methionine and separated on a 12% SDS polyacrylamide gel. Mutations in zinc fingers 3 and 4 disrupt binding of ZF5 to the RRS. In vitro translated proteins were incubated with a ³²P-labeled RRS oligonucleotide probe, and DNA binding was analyzed by EMSA. ZF5 binds specifically to the RRS motif. Increasing molar ratios (1, 5, 25x for ZF5, and 1, 25x for Rep78) of unlabeled DNA fragments containing the RRS or a mutant RRS were added as competitors. The tagged ZF5-DNA complex can be supershifted by a Myc-specific antibody. Rep78, RepTZAD, and luciferase (Luc) were included as controls.

Whereas wild-type ZF5 shifted a substantial amount of the RRS probe (BZ), ZF3 bound weakly, and no shift was detected for ZF5 4. These results were reproduced with epitope-tagged proteins. The specificity of DNA binding was shown by competition with increasing amounts of unlabeled probe containing either a wild-type or mutant RRS motif. Binding of ZF5 C, ZF5, and Rep78 to the RRS probe was blocked by excess amounts of wild-type RRS but not by mutant competitor. Epitope-tagged protein-DNA complexes were supershifted by an antibody. The in vitro analysis demonstrates that ZF5 binds to the RRS in a sequence-specific manner and that zinc finger 4 is crucial for DNA binding.

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention. As such, these changes and modifications are intended to be within the full range of equivalence of the following claims.

We claim:

1. A method for identifying molecules that interact with an adeno-associated viral regulatory element comprising;
a) contacting one or more molecules with a nucleic acid comprising one or more adeno-associated viral regulatory elements; and
b) identifying whether the one or more molecules are bound to the viral regulatory elements.

2. The method of claim 1 wherein the one or more molecules are proteins.

3. The method of claim 2, wherein the proteins comprise cellular proteins.

4. The method of claim 2, wherein the proteins comprise recombinant proteins.

5. The method of claim 2, wherein the proteins comprise synthetic proteins.

6. The method of claim 2, wherein the proteins are exogenous proteins.

7. The method of claim 1, wherein the one or more molecules are selected from the group consisting of peptides, antibodies, nucleic acids, lipids, carbohydrates and organic and inorganic compounds and various combinations thereof.

8. The method of claim 1, wherein the viral regulatory element comprises an inverted terminal repeat (ITR) or region thereof.

9. The method of claim 1, wherein the viral regulatory element comprises one or more copies a region of an ITR selected from the group consisting of regions A, A′, B, B′, C, C′ and D.

10. The method of claim 1, wherein the viral regulatory element comprises stem a A, stem B or stem C.

11. The method of claim 1, wherein the viral regulatory element comprises one or more copies of a Rep Recognition Sequence (RRS).

12. The method of claim 11, wherein there are at least two RRSs.

13. The method of claim 11, wherein the RRS comprises SEQ ID NO:1.

14. The method of claim 13, wherein the RRS is at least 90% homologous to SEQ ID NO: 1.

15. The method of claim 1, wherein the nucleic acid comprising the one or more adeno-associated viral regulatory elements further comprises a reporter gene positioned 3′ to the regulatory element and the detection of bound molecule is by detecting a product of the reporter gene.

16. The method of claim 15, wherein the product of the reporter gene is detected by a change in color.

17. The method of claim 16, wherein the reporter gene comprises a nucleic acid encoding beta-galactosidase.

18. The method of claim 1, wherein the nucleic acid comprising said one or more adeno-associated viral regulatory elements is integrated into a yeast genome.

19. The method of claim 1, comprising a nucleic acid comprising the viral regulatory element operably linked with a nutritional reporter gene whereby the bound molecule is detected by the ability to grow under selective conditions.

20. The method of claim 15, wherein the nucleic acid comprising the adeno-associated viral regulatory element further comprises a nutritional reporter gene positioned 3′ to the regulatory element and the detection of bound molecule is by the ability to grow under selective conditions.

21. The method of claim 19 or 20, wherein the nutritional reporter gene is HIS3.

22. The method of claim 19 or 20, wherein the nutritional reporter gene is selected from the group consisting of LEU2, TRP1, LYS2, MET15, URA3, ADE2, and ADE8.

23. The method of claim 2, wherein the proteins are derived from a cDNA library.

24 The method of claim 2, wherein the proteins are derived from a cellular lysate.

25. The method of claim 2, wherein the proteins are human proteins.

26. The method of claim 1, wherein identifying whether the molecules are bound to the viral regulatory elements is by a method selected from the group consisting of: virus product formation, 2D gel electrophoresis, electrophoretic mobility shift assay, immunoprecipitation, bimolecular interaction assay (BIAcore), affinity chromatography, and a two-hybrid assay.

27. A kit for analyzing the interaction between molecules and an adeno-associated viral regulatory element comprising;
a) a nucleic acid comprising the adeno-associated regulatory element; and
b) a reporter gene positioned 3′ to the adeno-associated regulatory element, wherein the detection of bound molecule is by detecting a product of the reporter gene.

28. The kit of claim 27, wherein the adeno-associated viral regulatory element comprises an inverted terminal repeat (ITR) or one or more regions thereof.

29. The kit of claim 27, wherein said viral regulatory element further comprises one or more copies of a Rep Recognition Sequence (RRS).

30. The kit of claim 29, wherein said one or more copies of a Rep Recognition Sequence (RRS) are in tandem.

31. The kit of claim 29, wherein said RRS comprises SEQ ID NO: 1.

32. The kit of claim 27, wherein said viral regulatory element is derived from adeno-associated virus.
